(19) United States
(12) Patent Application Publication
(10) Pub. No.: US 2025/0194945 A1
(43) Pub. Date: Jun. 19, 2025

(54) Endoscope Having Articulating Head With Axially Situated Electrodes

(71) Applicant: Diversatek Healthcare, Inc., Milwaukee, WI (US)

(72) Inventor: Charles L. Lindsay, Monument, CO (US)

(21) Appl. No.: 18/846,822

(22) PCT Filed: Mar. 14, 2023
(86) PCT No.: PCT/US2023/015196

§ 371 Date: Sep. 13, 2024

Related U.S. Application Data
(60) Provisional application No. 63/319,651, filed on Mar. 14, 2022.

(51) Int. Cl. A61B 5/0538; A61B 1/00; A61B 1/05; A61B 1/273; A61B 1/31; A61B 5/00

(52) U.S. Cl. CPC A61B 5/0538; A61B 1/00097; A61B 1/00137; A61B 1/05; A61B 1/273; A61B 1/31; A61B 5/4255; A61B 5/6847; A61B 5/7264; A61B 2560/0468; A61B 2562/046

(57) ABSTRACT

A mucosal impedance measuring device for measuring a pressure-controlled impedance of mucosa includes an endoscope, more specifically a duodenal scope, having an elongated body extending to a scope end in which the scope end is articulable relative to the elongated body and further having a plurality of impedance measuring electrodes disposed on an axial-most face of the scope end. The plurality of impedance measuring electrodes disposed on an axial-most face of the scope end can be placed into contact with mucosa, especially in the duodenum or colon, to establish the health, integrity, and/or condition of the mucosa by taking impedance measurements of the mucosa.

### CROSS-REFERENCE TO RELATED APPLICATIONS

This application claims the benefit of U.S. Provisional Patent Application No. 63/319,651 entitled “Endoscope Having Articulating Head with Axially Situated Electrodes” filed Mar. 14, 2022, which is hereby incorporated by reference for all purposes as if set forth in its entirety herein.

### STATEMENT REGARDING FEDERALLY SPONSORED RESEARCH

Not applicable.

### BACKGROUND

Recently, links between the electrical properties of esophageal mucosal tissues and the health of these esophageal mucosal tissues have been identified and investigated. Specifically, it has been determined that by the taking impedance measurements of the esophageal mucosa, that the condition or health of those esophageal mucosa can be determined. For example, patients with gastroesophageal reflux disease (GERD) have damaged mucosa in regions of their esophagus that are evident when the impedance measurements of the damaged mucosa are compared to the measurements of healthy, undamaged mucosa. Importantly, such impedance measurements are taken with the impedance measuring electrodes being placed in controlled contact with the esophageal mucosa under regulated pressure. By ensuring good pressure contact between the electrodes and the esophageal mucosa, meaningful impedance readings can be obtained which closely correspond to the health of the tissues based on empirical data.

With a new-found understanding this relationship between the impedance measurements of the esophageal mucosal and the health of the esophageal mucosa, unique devices have been designed to take such health-indicating impedance measurements. For example, U.S. Pat. No. 9,814,408 issued on Nov. 14, 2017 and U.S. Pat. No. 10,321,867 issued on Jun. 18, 2019, both of which are incorporated by reference for all purposes as if set forth in their entirety herein, disclose systems and methods involving the use of catheters and catheter systems that take pressure regulated impedance measurements of esophageal mucosa. Such devices are described as, for example, including catheters with associated balloons and impedance measuring electrodes which might be arranged in arrays or lines. During use of such a device, the patient is intubated with the catheter, the balloon is inflated to draw the electrodes into predicable and controlled contact with the esophageal mucosa, and impedance measurements are taken through the electrodes while the balloon remains inflated. The measured impedance can then be evaluated and compared to the known impedance values of heathy or unhealthy tissue. Furthermore, systems and methods have also been specifically disclosed in which an endoscope with an articulating scope end is used to press the electrodes against the mucosa. See for example, PCT Patent Application Publication No. 2020/0176508 published on Sep. 3, 2020, which is incorporated by reference for all purposes as if set forth in its entirety herein. In such design, instead of a balloon or bladder generating the pressure, the end of the scope may be articulated such that the sides of the end carrying the electrodes contact the mucosa.

Such devices are contemplated as permitting much faster identification of esophageal conditions as opposed to more traditional studies, which can last for multiple hours or even a few days.

### SUMMARY OF THE INVENTION

While catheterized systems of the type described above have been demonstrated to be able to collect useful impedance measurements of esophageal mucosa for time-efficient evaluation of esophageal mucosal health, such systems have been somewhat limited in their field of application. It has been established that such impedance measurements could be used to interrogate the integrity of mucosal tissue via impedance measurements in the esophagus using a balloon catheter and, in subsequent work, to evaluate either the esophagus or lower portions of the gastrointestinal tract such as the colon using an articulating scope head design in which the electrodes on radial or lateral sides of an articulating head of the endoscope in which the articulation of the end draws the electrodes into contact with the mucosa.

However, such systems have employed radially- or laterally-disposed electrodes on the catheter or endoscope with some associated structure for creating pressure controlled contact of the electrodes against the mucosa. Such design conceptions can potentially be problematic in that fluids can become trapped between the electrodes and the mucosa adversely impacting the quality of the impedance readings (potentially requiring multiple deployments to collect reliable readings or further assessment and selective exclusion of bad readings) and/or it may be difficult for the operator to visually identify the mucosa being measured.

Still further, in certain studies it has been perceived as necessary to map lengths of the gastrointestinal tract (for example, the lower esophagus) to provide sufficient data to support a diagnosis [for example, of GERD, Non-Erosive Reflux Disease (NERD), and eosinophilic esophagitis (EoE)]. While impedance-based studies are far shorter in duration than earlier studies not employing impedance, mapping entire regions does relatively increase the study time and further involves complex processing and assessment of data sets to come to a conclusion or assessment about the results of the study.

While such detailed interrogations and mapping of impedance over regions of mucosa have proven effective in supporting certain diagnoses, those involved in making the instant disclosure believe that, as in the esophagus, there may be a connection between mucosal damage in the duodenum and various small bowel disorders. Accordingly, this emerging field of diagnostics based on mucosal impedance and researchers and physicians operating in this field could benefit greatly from having a small device capable of easily measuring mucosal impedance in the duodenum. However, due to folding or raised surfaces found in the tissue in the duodenum and colon, existing mucosal impedance measuring devices with radially- or laterally-disposed electrodes are not particularly well-adapted for spot checks of mucosa in the duodenum.

To address this possibility and help researchers easily measure the mucosal impedance in the duodenum, a device is disclosed herein for spot checking the impedance of mucosa that will allow researchers to measure mucosal impedance quickly and accurately in the duodenum while preserving their ability to do further examination and biopsies. This low-profile device securely attaches to the tip of a duodenal scope and can easily be placed directly on a specific point of interest to provide a fast and reliable mucosal impedance measurement.

In this disclosed device, measurement accuracy can be achieved by using a factory-calibrated device placed on the tip on a duodenal scope. The device slightly protrudes past the tip of the scope, minimally impeding the camera's field of view while allowing researchers to easily, visualize guide the sensors into place for measurement. In addition, the system electronically monitors and reports the quality and stability of each impedance sensors on the device, giving the researcher real-time graphical feedback of the quality of their measurement. These features provide the researcher with the confidence needed to ensure measurements are done safely, quickly, and with the highest quality possible.

According to one aspect, a mucosal impedance measuring device is disclosed for measuring a pressure-controlled impedance of mucosa, especially in the duodenum. The device includes an endoscope, or more specifically a duodenal scope, having an elongated body extending to a scope end in which the scope end is articulable relative to the elongated body. The device further includes a plurality of impedance measuring electrodes disposed on an axial-most face of the scope end.

In some forms, the impedance measuring electrodes may be arranged to be placed in contact with mucosa during an endoscopy procedure.

In some forms, the endoscope may include a camera on a recessed axial face on the scope end. The camera may be disposed to view an area in front of the axial-most face of the scope end, thereby providing a visual indication of the mucosa the impedance measuring electrodes specifically contacts.

In some forms, the impedance measuring electrodes may include linear surfaces or segments which are positioned for contact with the mucosa.

In some forms, an angular extent of the axial-most face of the endoscope having the impedance measuring electrodes disposed thereon exceeds 180 degrees.

In some forms, the impedance measuring electrodes may each be U-shaped, having an axially forwardmost central segment that constitutes a mucosa contact surface of the respective impedance measuring electrodes.

In some forms, the plurality of impedance measuring electrodes may include four impedance measuring electrodes. The four impedance measuring electrodes can be two sets of electrode pairs in which each electrode pair constitutes a sensor (such that there are two sensors total). These pairs of electrodes can be positioned on opposite sides of the axial tip of the device such that the center of the paired electrodes are 180 degrees apart.

In some forms, the plurality of impedance measuring electrodes may be attached to the scope end via a cap secured in the axial end of the endoscope.

In some forms, the device may further include an impedance measuring system in electrical communication with the plurality of impedance measuring electrodes in which the impedance measuring system is configured to direct a current between the plurality of impedance measuring electrodes and through the mucosa and to measure the pressure-controlled impedance of the mucosa. The impedance measuring system may include software configured to determine whether the pressure-controlled impedance of the mucosa is a stable impedance measurement indicative of consistent pressure-regulated contact between the plurality of impedance measuring electrodes and the mucosa.

According to another aspect, a method is provided of measuring a pressure-controlled impedance of mucosa using the mucosal impedance measuring device as described above and elsewhere herein. The scope end of the endoscope is advanced in a patient to draw the impedance measuring electrodes situated at the axial-most face of the scope end into contact with the mucosa. After the impedance measuring electrodes contact the mucosa, a current is conducted between the plurality of impedance measuring electrodes and through the mucosa and measuring the pressure-controlled impedance of the mucosa.

In some forms, the method may involve using a camera on a recessed axial face on the scope end to view the mucosa and to direct the plurality of impedance measuring electrodes into contact with a region of the mucosa of interest for the measuring pressure-controlled impedance of the mucosa.

In some forms, the step of measuring the pressure-controlled impedance may involve using an impedance measuring system in electrical communication with the plurality of impedance measuring electrodes in which the impedance measuring system directs the current between the plurality of impedance measuring electrodes and through the mucosa and measures the pressure-controlled impedance of the mucosa.

In some forms, the mucosa at which the pressure-controlled impedance may be measured is located in the duodenum or colon.

These and still other advantages of the invention will be apparent from the detailed description and drawings. What follows is merely a description of some preferred embodiments of the present invention. To assess the full scope of the invention, the claims should be looked to as these preferred embodiments are not intended to be the only embodiments within the scope of the claims.

### BRIEF DESCRIPTION OF THE FIGURES

FIG. 1 is a perspective view of a mucosal impedance measuring device in which a set of impedance measuring electrodes are positioned on a front axial face of an endoscope or duodenal scope and a cameral is shown recessed into a cap supporting the electrodes.

FIG. 2 is a side view of the mucosal impedance measuring device of FIG. 1.

FIG. 3 is a front axial view of the mucosal impedance measuring device of FIG. 1.

FIG. 4 is an exploded perspective view of the mucosal impedance measuring device of FIG. 1 with the cap exploded off.

FIG. 5 is another exploded perspective view of the mucosal impedance measuring device of FIG. 1 which is similar to FIG. 4 but taken at a different angle and showing the electrodes exploded radially from the device.

FIG. 6 is a side view of the mucosal impedance measuring device of FIG. 1 in which the device is shown after being drawn into contact with mucosa for measuring the impedance of the mucosa.

### DETAILED DESCRIPTION

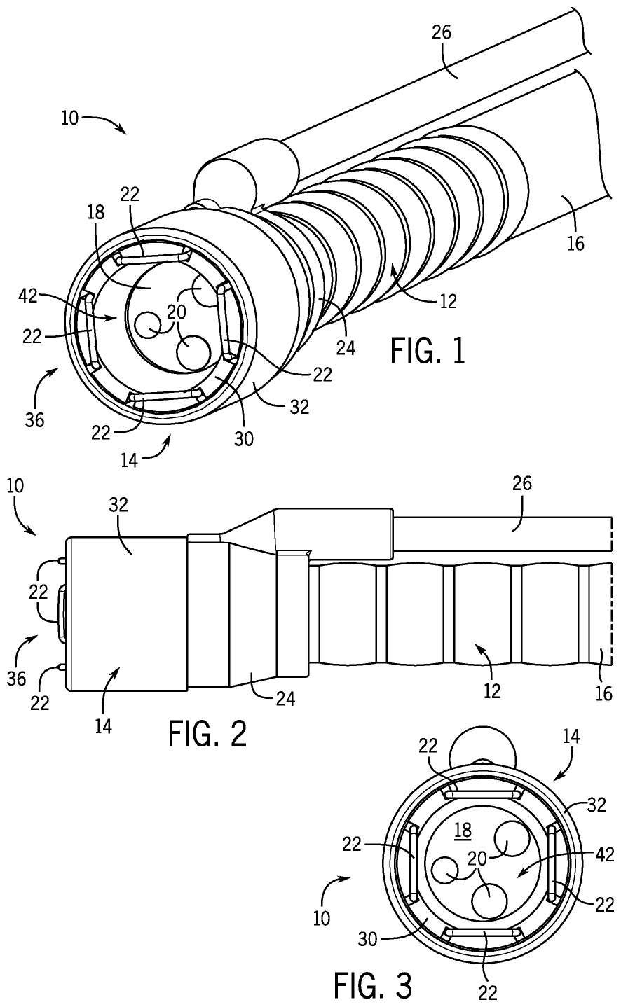

Referring to FIGS. 1 through 5, a mucosal impedance measuring device 10 and its constituent parts are illustrated. As illustrated, the mucosal impedance measuring device 10 includes an endoscope 12 in the form of a duodenal scope. An accessory 14 attached to the endoscope 12 for electrical interrogation of mucosa as will be described in greater detail below. It is noted that, while the endoscope 12 and the cap accessory 14 are illustrated as separate components in the illustrated embodiment, it is contemplated that the structure of the cap accessory 14 might in other forms be integrated directly into the structure of an endoscope 12 and such combined structure is contemplated under the scope of this disclosure rather than the illustrated multi-component construction. Put differently, the so-named “accessory” or cap could be part of the endoscope itself.

The endoscope 12 has an elongated body 16 extending to a scope end 18. Such an endoscope as endoscope 12 is generally known from the state of the art and the scope end 18 typically includes a camera and light on the axial end thereof for producing images of the inside of a patient or subject. As can be best seen in FIGS. 1, 3, and 5, there are multiple channels 20 which can be outfitted with light sources, cameras, or so forth or serve as irrigation or working channels, for example, which can be schematically understood as being present in the channels 20. The scope end 18 is articulable relative to the rest of the elongated body 16. This articulation both can be used to guide the endoscope 12 as it is inserted into the patient or subject and to direct the camera on the scope end 18 to capture video and/or images of interest within the body. The control of the articulation as well as the insertion/withdrawal of the endoscope 12 is typically manually performed by an operator such as a physician or technician to ensure that the scope end 18 is navigated appropriately.

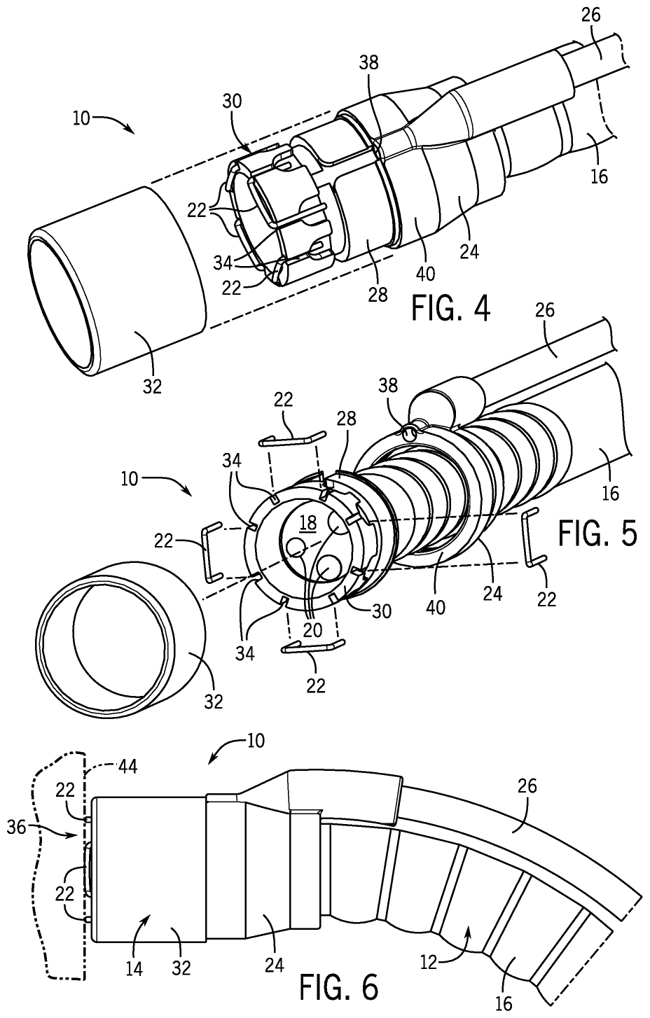

In the form illustrated, the accessory 14 is supported by or mounted onto a forwardmost portion of the endoscope 12 at the scope end 18, which here is generally tubular or cylindrical in shape. The as-mounted condition of the accessory 14 is illustrated in FIGS. 1-3, while FIGS. 4 and 5 illustrate a partially exploded views to show the constituent parts of the accessory 14.

From FIGS. 4 and 5 in particular, the cap accessory 14 may be a multi-part assembly including a plurality of impedance sensing electrodes 22, a rear mounting sheath 24 which can attach to a channel 26 for passage of conductors that are connected to the impedance sensing electrodes 22, an inner sleeve 28, an electrode-supporting insert 30, and an outer sleeve or cap 32.

As can be seen in FIGS. 4 and 5, the plurality of impedance sensing electrodes 22 are U-shaped—resembling standard staples in shape with slightly rounded bends at the corners—and are received in slots 34 on the outer radial side of the electrode-supporting insert 30. The arrangement of the electrodes 22 within the slots 34 are such that the bottom of the U-shape is positioned axially forward and the two legs of each electrode 22 extend axially backward. In the illustrated embodiment, the bottom of the U-shape of the impedance sensing electrodes 22 are ultimately disposed on a forward-most axial end 36 of the cap accessory 14 and (as-mounted) the device 10 for coming into contact with mucosa during use.

There are four impedance measuring electrodes 22 in the embodiment depicted and which each have a linear contacting surface. Pairs of the electrodes 22 can constitute an impedance sensor formed by the electrode pair, such that the four electrodes 22 provide two impedance sensors. As depicted, the electrodes 22 opposing one another are paired to form an electrode pair or impedance sensor. By stating that the electrodes 22 are opposing, it is meant that these electrode pairs can have centers spaced approximately 180 degrees from one another around the circumference of the axial tip. This opposing arrangement of the electrodes pairs means that the mucosa that is disposed between the electrodes 22 during contact of the tip against the mucosa can be measured at least twice and from the two different sets of electrode pairs, which are offset 90 degrees from one another in the form illustrated. However, other numbers of electrodes may be present in other designs and the contacting surfaces for contact against the mucosa could have a profile different than those illustrated. For example, the contacting surfaces could be arcuate or shaped differently. It is noted that, in the illustrated form, the electrodes 22 are evenly angularly spaced around the periphery of the axial end 36 of the cap accessory 14, with their centers are spaced 90 degrees from one another with small gaps between the ends of the electrodes 22, and such that, if one extends angularly around the axial end 36 of the cap accessory 14 a full 360 degrees, the electrodes 22 are present over more than 180 degrees of that 360 degree angular distance. So unlike electrodes that may have smaller pads or point contact, with the exposed lengths on their ends, each of the electrodes 22 are more likely to make good contact with mucosa they may be contacted against for taking an impedance measurement of the mucosa.

Without being limited to any particular design, one or more of the rearward-most axial ends of each of the electrodes 22 in this design can be attached to or placed in electrical communication with a corresponding conductor such as a wire (not shown). Such connection can be either direct (for example, by soldiering or a connector) or indirect (for example, pressed against a contact plate which is in turn connected to the conductor or wire). In any event, such conductors may extend rearward out of the accessory 14 through the channel 26 by being routed to and through an opening 38 of the rear mounting sheath 24 such that the conductors can be connected to an impedance measuring system at the other end of the endoscope 12, which is outside the patient's body during intubation.

The cap accessory 14 can be mounted to the endoscope 12 in a number of ways, one of which is now described. The sheath 24 may be placed on the endoscope 12 such that a ring 40 of the sheath 24 circumferentially encircles the end of the endoscope 12 and the channel 26 attached thereto extends axially backward therefrom (i.e., away from the scope end 18). The electrode-supporting insert 30 may be received on the forward axial end of the inner sleeve 28 as best depicted in FIG. 4 and positioned near the forward axial end of the endoscope 12. The insert 30 and the inner sleeve 28 may then be secured to the endoscope 12 by attaching the rear end of the outer cap 32 to the forward end of the sheath 24, capturing the insert 30 and the inner sleeve 28 therebetween. It is contemplated that the outer cap 32 may be secured to the sheath 24 in a number of ways including reversible or irreversible connections, such as for example, by screwing threads into one another, by forming an interference fit, by joining via adhesive, and so forth. While such connection of the sheath 24 and the outer cap 32 may alone axially position the accessory 14 with respect to the scope end 18 of the endoscope 12 by virtue of features of the sheath 24 and/or outer cap 32 with the endoscope 12 or the scope end 18, it is also contemplated that the drawing of the outer cap 32 to the sheath 24 may create a compression fit. Such compression fit could be formed, for example, axially drawing the cap 32 and sheath 24 together such that the insert 30 and inner sleeve 28 are forced together by this attachment action of the cap 32 and sheath 24. One or both of the sleeve 28 and insert 30 may have an axial split and/or a ramped periphery such that bringing the sleeve 28 and insert 30 together causes one or both of the parts to have an inner periphery which constricts to as they are mechanically brought together into interference with one another, thereby securing the constricted inner periphery of the accessory 14 to the outer periphery of the endoscope 12.

As best seen in FIGS. 1, 2, and 6, when the accessory 14 so assembled and is secured to the endoscope 12, the electrodes 22 are positioned most forwardly at the axial end 36. Moreover, the front end of the endoscope 12 containing the channels 20 having the camera, light, working passage, and so forth is axially offset to form a recess 42 relative to the axial end 36 of the accessory 14 which supports the electrodes 22. This creates an arrangement and construction in which the camera can be used to direct the endoscope 12 and view the mucosal tissue in front of the axial end 36 of the cap accessory 14, even though the camera is recessed. Moreover, keeping the camera recessed prevents the camera from directly contacting the tissue and thus helps keep the camera lens clean. Further, when the electrodes 22 of the cap accessory 14 are brought into contact with the mucosa, there is the opportunity to visually interrogate the mucosa as well as visually ensure good contact between the electrodes 22 and the mucosa.

FIG. 6 schematically illustrates the in situ use of the device 10 to measure the impedance of a hypothetical mucosa 44, and in particular the mucosa 44 in the duodenum of a patient or subject. The endoscope 12 is first directed through the subject's body to the site of interest. In this case, the endoscope 12 may be directed through the esophagus and into the duodenum. In this position, the axial end of the cap accessory 14 and the electrodes 22 specifically are brought into contact with the mucosa 44 as depicted schematically in FIG. 6. Ideally, all electrodes 22 are brought into contact with the mucosa 44 by dead-ending the axial tip 36 of the device with the mucosa 44. As mentioned above and as a matter of process, the endoscope 12 can be directed using a camera and light or the like, and so the mucosa 44 being contacted can be visually seen prior to contacting with the electrodes 22. This contact between the electrodes 22 and the mucosa 44 is to be made under regulated or controlled pressure of the electrodes 22 against the mucosa 44 which may be controlled by the action of advancing the endoscope 12 and/or articulating the scope head or end 18. After the electrodes 22 are in pressure-controlled contact with the mucosa 44, then an impedance measuring system in electrical communication with the electrodes 22 (via conductors or wires) can be used to control the flow of a current through the conductors into the electrodes 22 in order to measure the impedance of the mucosa 44 between sets of the electrodes 22. This electrical interrogation may be done in a pre-programmed manner or manually directed sequence between various ones of the electrodes 22. Moreover, the impedance may be measured in a number of ways between the individual electrodes 22 and in various permutations across them to robustly characterize the spot of contact and interrogation (i.e., from one electrode to an adjacent electrode and/or from one electrode to an electrode on an opposing sides). The impedance measuring system can include a processor, controller, a current source, and circuitry for measuring the impedance between the impedance measuring electrodes 22. For example, it can include a current production source which can be used to run current from one electrode to another (and through mucosa, as will be described below) and circuitry for measuring the impedance between the electrodes (and, again, of the mucosa therebetween).

It is contemplated that the impedance measuring system can not only include electronics for obtaining impedance measurements, but also software and/or hardware for determining whether the measured impedance measurements are valid. Since, as explained above and from the patent and application incorporated by reference, the impedance measurements of mucosa are only valid if taken under controlled pressure and are sufficiently stable (meaning that good consistent contact is made between the electrodes and the mucosa), it is contemplated that the impedance measuring system can include testing logic to evaluate and confirm with the end user whether an obtained impedance measurement of mucosa is a good and valid measurement or includes stability issues or absolute impedance values that are indicative of an improper reading due to bad or inconsistent contact between the electrodes and the mucosa. For example, it is contemplated that impedance might be measured over a predetermined window of time (perhaps, a few hundred milliseconds or various seconds) and the signal of the impedance measured over time analyzed to determine whether the impedance is stable and within expected ranges for either healthy or unhealthy mucosa.

Still further, it is contemplated that there could be pressure-sensing elements attached to electrodes or between the electrodes that independently and electronically confirm stable pressure contact exists between the electrodes 22 and mucosa 44 when the electrodes 22 are dead-ended against the mucosa 44. Such pressure-sensing elements and information therefrom might be used separately from or in combination with the software/hardware analysis of the impedance signal to assess the validity of an impedance measurement.

In some instances, a visualization of the measured data may be provided to help assess the quality and/or consistency of the obtained measurements. When graphically depicting the mucosal impedance on the visual depiction, it is considered that the impedance readings may be color-coded to improve understanding by the viewer. For example, measurements that are taken as being indicative of healthy tissue may be depicted as green, while unhealthy tissue depicted as red. Still further, color gradients could be used to depict either the magnitude of the reading (e.g., light green for marginally healthy tissue and dark green for strongly healthy tissue.

Still further, it is contemplated that apart from merely using color and/or number and even spatially mapping the reading, it is possible that quality of the reading (e.g., the stability of the reading) might also be mapped physically on the visualized depiction. In this way, the operator may also be able to better assess whether certain regions were more difficult than others to collect measurements from and, as a result of this difficultly in collection, if the measurements from that area may need to be recollected or be examined more closely or skeptically. Still further, it may be possible to provide other layers not just for health of tissue base on impedance or quality of reading, but for other conditions of interest, such as for example the average diameter of the localized region of the colon if such information has been collected.

Returning now to the structure of the endoscope and accessory for the endoscope, it is contemplated that in addition to the structures and methods described above, various other modifications and additions might be feasible to facilitate authentication of the device and/or ensure proper use. As one example, when the device is provided as an accessory to an endoscope, the accessory structure might house a small chip (for example, in the structure of the support) which could provide authentication information to the impedance measuring system to confirm that the accessory device is genuine and/or provide calibration information relating to the specific accessory so that, when that calibration information is accounted for by the impedance measuring system, the impedance measurements taken with the accessory are accurate. Each device may be factory calibrated to ensure each device has a consistent impedance point of reference that is consistent to the balloon device. Still further, in such case that the impedance measuring electrodes are tightly integrated into the endoscope, such authentication information and/or calibration data might similarly be housed in the structure of the endoscope. It is also contemplated that such a chip or memory could store information about the number of times that the accessory (or endoscope, if integrated) has been used to ensure both that the accessory (or endoscope) is being properly used and has not been fouled in some way since its calibration. For example, the device may be engineered for one-time use or N-time uses and the chip may hold information about whether the use or N-time uses has occurred or not; it is contemplated that, if the use or uses have occurred, then the impedance measuring system may provide the user with an indication that the device cannot be used without first replacing the accessory (or endoscope, if integrated).

In sum, a device is disclosed in which impedance sensing electrodes are situated at the axial end of an endoscope for measuring the mucosa during an endoscopy. In particular, this device can be useful for navigating the endoscope to the point of interest and then measuring the impedance of the mucosa in a spot test especially in the duodenum (although this device could be helpful in measuring impedance at other locations as well, including, but not limited, to the colon).

The present disclosure has described one or more preferred embodiments, and it should be appreciated that many equivalents, alternatives, variations, and modifications, aside from those expressly stated, are possible and within the scope of the invention.

What is claimed is:

1. A mucosal impedance measuring device for measuring a pressure-controlled impedance of mucosa, the device comprising:
an endoscope having an elongated body extending to a scope end in which the scope end is articulable relative to the elongated body; and
a plurality of impedance measuring electrodes disposed on an axial-most face of the scope end.

2. The mucosal impedance measuring device of claim 1, wherein the impedance measuring electrodes are arranged to be placed in contact with mucosa during an endoscopy procedure.

3. The mucosal impedance measuring device of claim 1, wherein the endoscope includes a camera on a recessed axial face on the scope end.

4. The mucosal impedance measuring device of claim 3, wherein the camera is disposed to view an area in front of the axial-most face of the scope end, thereby providing a visual indication of the mucosa the impedance measuring electrodes specifically contacts.

5. The mucosal impedance measuring device of claim 1, wherein the impedance measuring electrodes include linear surfaces which are positioned for contact with the mucosa.

6. The mucosal impedance measuring device of claim 1, wherein an angular extent of the axial-most face having the impedance measuring electrodes disposed thereon exceeds 180 degrees.

7. The mucosal impedance measuring device of claim 1, wherein the impedance measuring electrodes are each U-shaped having an axially forwardmost central segment that constitutes a mucosa contact surface of the impedance measuring electrodes

8. The mucosal impedance measuring device of claim 1, wherein the plurality of impedance measuring electrodes include four impedance measuring electrodes.

9. The mucosal impedance measuring device of claim 1, wherein the plurality of impedance measuring electrodes are attached to the scope end via a cap secured in the axial end of the endoscope.

10. The mucosal impedance measuring device of claim 1, further comprising an impedance measuring system in electrical communication with the plurality of impedance measuring electrodes in which the impedance measuring system is configured to direct a current between the plurality of impedance measuring electrodes and through the mucosa and to measure the pressure-controlled impedance of the mucosa.

11. The mucosal impedance measuring device of claim 10, wherein the impedance measuring system includes software configured to determine whether the pressure-controlled impedance of the mucosa is a stable impedance measurement indicative of consistent pressure-regulated contact between the plurality of impedance measuring electrodes and the mucosa.

12. A method of measuring a pressure-controlled impedance of mucosa using the mucosal impedance measuring device of claim 1, the method comprising:
advancing the scope end of the endoscope within a patient to draw the impedance measuring electrodes situated at the axial-most face of the scope end into contact with the mucosa; and
conducting a current between the plurality of impedance measuring electrodes and through the mucosa and measuring the pressure-controlled impedance of the mucosa.

13. The method of claim 12, further comprising using a camera on a recessed axial face on the scope end to view the mucosa and to direct the plurality of impedance measuring electrodes into contact with a region of the mucosa of interest for the measuring pressure-controlled impedance of the mucosa.

14. The method of claim 12, wherein the step of measuring the pressure-controlled impedance involves using an impedance measuring system in electrical communication with the plurality of impedance measuring electrodes in which the impedance measuring system directs the current between the plurality of impedance measuring electrodes and through the mucosa and measures the pressure-controlled impedance of the mucosa.

15. The method of claim 12, wherein the mucosa at which the pressure-controlled impedance is measured is located in the duodenum or colon.
